(19) United States
(12) Patent Application Publication
(10) Pub. No.: US 2016/0377588 A1
(43) Pub. Date: Dec. 29, 2016

(54) Sensor device for determining the evaporation pressure of a fluid, in particular of a fluidic fuel

(71) Applicant: Hella KGaA Hueck & Co., Lippstadt (DE)

(72) Inventors: Torsten Eggers, Bremen (DE); Hagen Muller, Bad Wunnenberg (DE); Thomas Niemann, Delmenhorst (DE)

(21) Appl. No.: 15/197,084

(22) Filed: Jun. 29, 2016

(30) Foreign Application Priority Data
Jun. 29, 2015 (DE) 10 2015 008 300.7

(51) Int. Cl. G01N 33/22; F02D 41/00

(52) U.S. Cl. CPC G01N 33/22; F02D 41/0025; B60K 2015/0321

(57) ABSTRACT

In a sensor device for determining the evaporation pressure of a fluid, in particular of a fluidic fuel, in a vehicle with at least one heating element, the invention provides that the sensor device has a filling level sensor with a circuit carrier and that the heating element is arranged on the circuit carrier of the filling level sensor.

### BACKGROUND OF THE INVENTION

Field of the Invention

The invention relates to a senor device for determining the evaporation pressure of a fluid, in particular of a fluidic fuel, in a vehicle with at least one heating element.

Brief Description of the Related Art

Devices for determining an evaporation pressure are, for example, used in motor vehicle engine controls. For example, fuel pumps are operated as needed in petrol-powered direct-injection engines, the feed rate of the fuel pumps thus being adapted to the actual need of the engine. Here, a sufficiently high fuel pressure in the fuel line needs to be ensured, when the fuel is pumped towards the engine, in order to prevent the fuel forming into vapour bubbles. Should such a formation of vapour bubbles in the fuel line occur due to higher engine temperatures or lower pressures, the engine is unable to start safely. The fuel pressure may thus not exceed the temperature-dependent vapour pressure. This is problematic, precisely in connection with start-stop automatic systems, as engine starting problems can occur in the event of overheated engines, as the necessary pre-pressure in the fuel line is crucially dependent on the fuel vaporization pressure. The fuel vaporization pressure is thus dependent on both the temperature and the proportion of the low-boiling fuel portions.

In order to address this problem, an assessment device for fuel properties of a combustion engine is known, for example, from DE 199 55 796 A1. The intention of the latter is to provide a fuel dosage adjusted to the respective fuel property by assessing the precise fuel properties. The assessment device has a crank angle sensor for generating a crank angle signal according to a rotation rate of the combustion engine, a starter switch for emitting a start signal at the time of the starting operation of the combustion engine and a device for detecting the combustion at the starting time of the combustion engine. A counting device calculates the number of times the crank angle signal is started up, said number then being compared with a standard number and thus inferring the fuel properties.

A disadvantage in the known devices is that the sensors for determining the fuel properties need to be separately integrated in the fuel line, for example. This involves additional assembly effort as well as additionally required components.

### SUMMARY OF THE INVENTION

The object underlying the invention is to propose a device, by means of which is enabled a particularly simple determination of the vaporization properties of a fuel and for which no additional components are required.

The object is achieved by a device with the features of claim 1. Advantageous configuration of the invention are provided in the dependent claims.

In a device for determining the vaporization pressure of a fluid, in particular of a fluidic fuel, in a vehicle with at least one heating element, the invention provides that the sensor device has a filling level sensor with a circuit carrier and that the heating element is arranged on the circuit carrier of the filling level sensor. In particular, the filling level sensor can be arranged in a fuel line or in a fuel container, in particular a tank, of a vehicle. A heating element is arranged on the fuel level sensor that enables the fluidic fuel to be heated. In particular, the heating element has an electrically conductive connection with the circuit carrier of the filling level sensor. For example, the fluidic fuel can be heated by the heating element with constant heating power, wherein the temperature change of the fluidic fuel is determined. The temperature change can, for example, be recorded by a temperature sensor, which is also arranged on the circuit carrier of the filling level sensor. If the fluidic fuel is heated up to such an extent that a formation of vapour bubbles occurs, this abruptly increases the heat dissipation. The abrupt increase in heat dissipation is caused by the incipient convection, which is attributable to the rising vapour bubbles. The temperature point at which the abrupt change in the heat dissipation occurs determines the boiling temperature of the fluidic fuel. Fluidic fuels are usually liquid mixtures so that the initial boiling point of the boiling range is determined. When the vapour bubbles start to form, the temporal temperature profile on the heating element changes, wherein the temperature on the heating element once the vapour bubbles have started to form initially remains almost constant or increases substantially slower than before the formation of the vapour bubbles, despite further continuous heating. The evaporation behaviour of the fluidic fuel can thus be characterized via the temperature at which the first vapour bubbles form. In addition to the use of a temperature sensor, the heating element can also be designed as a temperature-dependent resistor, and therefore the heating element itself can be used as a means for measuring temperature. The heating element can have an electrical and/or a signal-conducting connection to evaluation units or similar units of the filling level sensor. The integration of the heating element on the circuit carrier of the filling level sensor allows a considerable reduction in the component list and in the assembly effort to be achieved, in that already available components are used.

The sensor device has at least one flow-directing element and said flow-directing element is arranged in the area of the heating element. Said heating element allows the fluid in the area surrounding the heating element to be heated until such time as vapour bubbles form. The resulting vapour bubbles rise in the fluid, so that convection occurs. The recording to the associated increased heat transfer allows the boiling point of the fluid to be inferred. In order to enable a precise measurement here, at least one flow-directing element is arranged in the area of the heating element. The flow-directing element wards off transverse flows, which emerge in the fluid container and may disturb the heat transfer, from the position of the heating element, thus allowing an exact measurement to be made. At least one flow channel is designed between the flow-directing element and the circuit carrier on which the heating element is arranged. The formation of a flow channel allows the resulting vapour bubbles to rise undisturbed in the fluid volume. A post-flow of a fluid volume with lower temperature is also possible, without any turbulence or other disruptions being caused by transverse flows.

In one embodiment of the invention the heating element has a ceramic supporting part. For example, the heating element can be a platinum heating element which is arranged on a ceramic supporting part, for example a ceramic chip, is arranged. The ceramic support results in a thermal decoupling of the heating element from the circuit carrier of the filling level sensor. The heating element can comprise both SMD components and components with leads for direct soldering onto the circuit carrier. In the embodiment as an SMD component the heating element can be used as a finished element with protective covering and supporting part.

In a further embodiment of the invention the sensor device is surrounded at least in sections by a protective covering made of a protective material and said protective covering has at least one recess in the area of the heating element. The protective covering protects the sensor device when measuring chemically aggressive fluids, such as fuels, for example. In order to enable a thermal connection between the heating element and the fluid for heating the fluid, the protective covering has a recess in the area of the heating element, in particular in the area of the heating coils. The protective covering can, for example, consist of a thermoset material.

In a design development of the invention the flow-directing element has a semi-tubular cross-section. The flow-directing element can be configured in a semi-tubular manner, in particular the longitudinal sides of the flow-directing element can have a connection to the surface of the circuit carrier or the protective covering, and therefore the heating element is shielded by the flow-directing element against turbulence. The semi-tubular cross-section of the flow-directing element and the connection of the longitudinal edges of the flow-directing element to the circuit carrier create a flow channel with semi-tubular cross-section. The heating element is thereby arranged directly adjacent to the flow channel.

In a design development of the invention the diameter of the semi-tubular cross-section of the flow-directing element has a ratio of approximately 1:5 relative to the length of the flow-directing element. Due to the fact that the length of the flow-directing element is approximately five times greater than the diameter thereof, a stack effect occurs during the heating of the fluidic fuel and creation of fuel vapour bubbles, said chimney effect supporting the transfer of the vapour bubbles. The measuring sensitivity of the arrangement is thus further increased.

In a design development of the invention the diameter of the semi-tubular cross-section of the flow-directing element equals approximately 4 mm and the length of said flow-directing element equals approx. 20 mm. The measurements of the flow-directing element ensure the formation of a stack effect for discharging the resulting fuel vapour bubbles.

In a further design development of the invention the flow-conducting element consists of a metallic material. A flow-directing element made of a metallic material, which is arranged in the area of the heating element, can serve as an explosion protection element due to the high thermal conductivity thereof. The gas mixture contained in the fuel vapour bubbles can have a fuel proportion of 3% to 20% and is thus ignitable. The high thermal conductivity of the flow-directing element allows the heat to be rapidly dissipated, thus allowing the temperature to be kept below the ignition point.

In a further design development, the heating element is arranged at the end of the circuit carrier facing away from the fluid level. The heating element is arranged at the end of the circuit carrier, i.e. of the fuel level sensor, which faces away from the fluid level, i.e. the filling level amount of the fluid. This ensures that the heating element is usually located below the fluid level, i.e. is covered by the fluid.

A further aspect of the invention relates to a motor vehicle with a sensor device according to the invention for determining the evaporation pressure of a fluidic fuel. In particular, motor vehicles have sensor devices for determining the filling level of a fluidic fuel. A heating element is arranged on the circuit carrier of the filling level sensor and connected with the electronic components of said filling level sensor, and therefore the filling level sensor can also serve as sensor device for determining the evaporation pressure of the fuel. The combination of the two sensor devices achieves a considerable reduction in the component list.

The invention is explained in more detail in the following on the basis of a preferred exemplary embodiment shown in the drawing. In detail, shown in:

### BRIEF DESCRIPTION OF THE DRAWINGS

FIG. 1 is a perspective view of a partially cut diagram of a sensor device according to the invention;

FIG. 2 is a perspective view of a partially cut diagram of a sensor device according to the invention surrounded by a protective covering; and

FIG. 3 is a perspective view of a schematic diagram of a sensor device according to the invention with a protective coat and a flow-directing element.

### DETAILED DESCRIPTION OF THE PREFERRED EMBODIMENTS

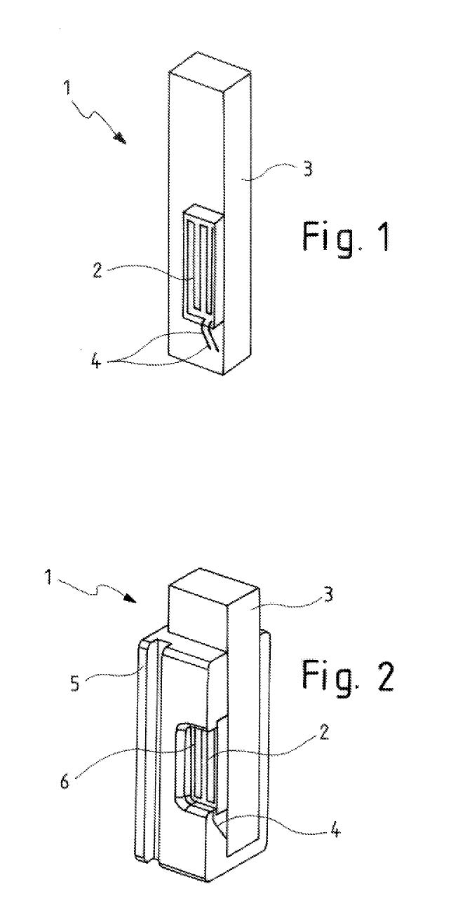

FIG. 1 shows a sensor device 1 with a heating element 2 and a circuit carrier 3. Further electronic components for forming a filling level sensor are arranged on the circuit carrier 3. The heating element 2 has a connection to the circuit carrier 3 and is in electrical contact with the circuit carrier via the bonding wires 4. Furthermore, the heating element 2 can be in electrical contact with, for example, an assessment unit, which is also arranged on the circuit carrier 3. The heating element 2 is arranged at the end of the circuit carrier, which is facing away from the fluid level of the fluid to be examined, and is therefore usually covered by fluid.

FIG. 2 shows a sensor device 1 according to FIG. 1. Identical components are indicated with identical reference numerals. The sensor device 1 has a protective covering 5 that can, for example, consist of a thermoset material. Said protective covering 5 has a recess 6, which is arranged in the area of the heating element 2. The recess 6 allows thermal contact between the heating element 2 and the fluid, so that the fluid can be heated by the heating element 2.

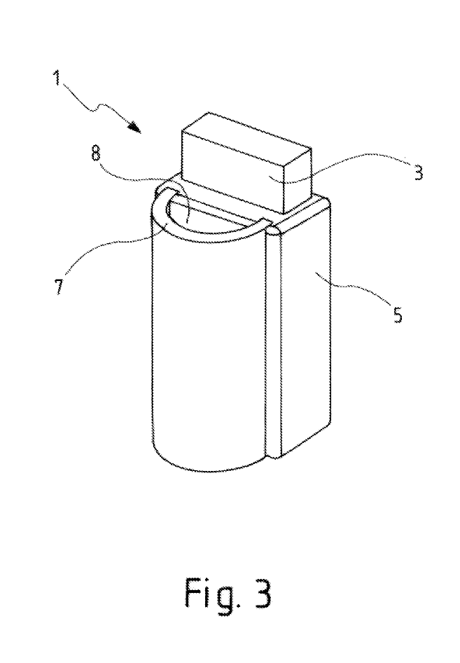

FIG. 3 shows a sensor device according to FIG. 2. Identical components are indicated with identical reference numerals. The sensor device 1 has a flow-directing element 7. Said flow-directing element 7 has a semi-tubular cross-section, the diameter of which has a ratio of 1:5 relative to the length of the flow-directing element 7. A flow channel 8 is designed between the flow-directing element 7 and the circuit carrier 3 covered by the protective covering 5. The flow-directing element 7 is formed of a metallic metal. Due to the high thermal conductivity of the metallic material, the flow-directing element 7 effects an explosion protection, in that heat dissipation keeps the temperature below the ignition temperature of the fuel vapour, which is located in the fuel vapour bubbles. The heating element 2 allows the fluidic fuel to be heated, in the tank of a motor vehicle, for example. A formation of fuel vapour bubbles may occur due to the continuous heating. Such fuel vapour bubbles rise in the fluidic fuel, thus ensuring more effective heat dissipation from the heating element 2. Such more effective heat dissipation can, for example, be determined on the basis of a no longer or not very strongly increasing temperature by means of a temperature sensor or can be directly determined via the heating element 2. The temperature at which fuel vapour bubbles occur is an indication of the boiling temperature of the fuel or the first boiling temperature of the fuel mixture. By the of the flow channel 8 formed through the flow-directing element 7 can lead to a stack effect, thus supporting the heat transfer and allowing a more precise measurement of the boiling temperatures.

All the features stated in the preceding description and in the claims can be combined on a purely discretionary basis with the features of the independent claim. The disclosure of the invention is thus not restricted to the described or claimed combinations of features; instead all expedient combinations of features within the scope of the invention should be considered as disclosed.

1. A sensor device for determining the evaporation pressure of a fluid, in particular of a fluidic fuel, in a vehicle with at least one heating element, wherein:
the sensor device has a filling level sensor with a circuit carrier, the heating element is arranged on the circuit carrier of the filling level sensor, the sensor device has at least one flow-directing element and said flow-directing element is arranged in the area of the heating element, and
at least one flow channel is formed between the flow-directing element and the circuit carrier on which the heating element is arranged.

2. The sensor device according to claim 1, wherein the heating element has a ceramic supporting part.

3. The sensor device according to claim 1, wherein the sensor device is surrounded at least in sections by a protective covering made of a protective material, and that the protective covering has at least one recess in the area of the heating element.

4. The sensor device according to claim 1, wherein the flow-directing element has a semi-tubular cross-section.

5. The sensor device according to claim 4, wherein the diameter of the semi-tubular cross-section of the flow-directing element is approximately 1:5 in ratio relative to the length of the flow-directing element.

6. The sensor device according to claim 1, wherein the diameter of the semi-tubular cross-section of the flow-directing element equals approximately 4 mm and the length of the flow-directing element equals approximately 20 mm.

7. The sensor device according to claim 1, wherein the flow-directing element consists of a metallic material.

8. The sensor device according to claim 1, wherein the heating elements is arranged at an end of the circuit carrier facing away from the fluid level.

9. A motor vehicle with a sensor device for determining the evaporation pressure of a fluidic fuel according to claim 1.
